(19) United States
(12) Patent Application Publication
(10) Pub. No.: US 2006/0183042 A1
(43) Pub. Date: Aug. 17, 2006

(54) Substituted oligofluorene for organic light-emitting diode and organic photoconductor

(76) Inventors: Wen-Yao Huang, Taoyuan City (TW); Yen-Cheng Tsai, Taipei City (TW); Jun-Lung Lee, Taipei (TW); Shih-Chien Chiu, Nanjhuang Township (TW); Chih-Hwa Cherng, Taipei City (TW); Chung-Chih Wu, Taipei City (TW); Wen-Yi Hung, Sanchong City (TW); Yu-Ting Lin, Wufong Township (TW); Tsung-Li Liu, Taipei City (TW); Ping-Yuan Hsieh, Taipei City (TW); Ken-Tsung Wong, Luzhou City (TW); Fu-Chuan Fang, Daliao Township (TW); Yuh-Yih Chien, Banciao City (TW); Chung-Feng Wang, Yuchih Township (TW); Ruei-Tang Chen, Kaohsiung City (TW); Teng-Chih Chao, Pingzhen City (TW); You-Ming Chen, Banciao City (TW)

Correspondence Address: Raymond Y. Chan, Suite 128, 108 N. Ynez Avenue, Monterey Park, CA, 91754, US

(21) Appl. No.: 11/060,636

(22) Filed: Feb. 16, 2005

(51) Int. Cl. G03G 5/06; G03G 5/047; C07F 7/02; C07C 13/48

(52) U.S. Cl. 430/58.05; 430/72; 546/14; 585/26

(57) ABSTRACT

A substituted oligofluorene for organic light-emitting diode (OLED) and organic photoconductor (OPC) has a chemical structure of: 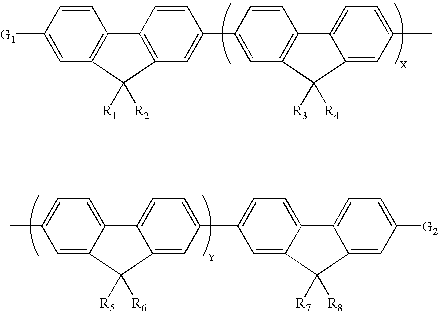
wherein X and Y are an integer of 0 or 1, G₁ and G₂ are independently of either CₙH₂ₙ₊₁ or CₙH₂ₙ₊₁O; and n is an integer of 0 to 16.

### BACKGROUND OF THE PRESENT INVENTION

1. Field of Invention

The present invention relates to substituted oligofluorenes and their use in the electronics industry.

2. Description of Related Arts

The use of organic compounds as active components (functional materials) has been reality and is expected in the future. Thus, charge transport materials based on organic compounds have been used for many years in xerography and laser printing. The use of specific semiconductive organic compounds, some of which are also capable of emission of light in the visible region of the spectrum, is just at the beginning of introduction onto the market, for example in organic electroluminescence devices. The use of organic charge transport layers in applications such as organic integrated circuits (organic ICs) and organic solar cells has already progressed a long way, at least in the research stage, so that introduction onto the market may be expected within the next few years. The number of further possibilities is very large, but frequently only as a modification of the above-described processes, as evidenced by the examples of organic solid-state laser diodes and organic photodetectors. In some of these modern applications, development has already progressed a long way, but there is still, depending on the application, a tremendous need for technical improvements.

Organic electroluminescence devices and their individual components, organic light-emitting diodes (OLEDs) have already been introduced on to the market, as evidenced by the commercially available products, such as automobile radios, digital cameras and cellular phones, having an “organic display” thereon. Further such products will be introduced shortly.

Nevertheless, considerable improvements are still necessary to make these displays truly competitive with the liquid crystal displays (LCDs) which dominate the market at present, or to make it possible for the LCDs to be overtaken.

The general structure of organic electroluminescence devices is described, for example, in U.S. Pat. No. 4,539,507 and U.S. Pat. No. 5,151,629. An organic electroluminescence device usually consists of a plurality of layers which are preferably applied overlappedly with each other by means of vacuum methods. These layers are specifically a support plate or substrate (usually glass or a plastic film), a transparent anode (usually indium-tin oxide, ITO), a hole injection layer (HIL), a hole transport layer (HTL) usually based on triarylamine derivatives, an emission layer (EML), an electron transport layer (ETL), an electron injection layer (EIL), and a cathode.

Accordingly, the hole injection layer is embodied as one based on copper phthalocyanine (CuPc), conductive polymers such as polyaniline (PANI) or polythiophene derivatives (e.g. PEDOT). The emission layer can sometimes coincide with the layers 4 or 6, but is usually made up of host molecules doped with fluorescent dyes or phosphorescent dyes. The electron transport layer is mostly based on aluminum tris-8-hydroxyquinoxalinate (Alq3). The electron injection layer can sometimes coincide with the layer 6 or a small part of the cathode is specially treated or specially deposited. The cathode is generally made of metals, metal combinations or metal alloys having a low work function, e.g. Ca, Ba, Mg, Al, In, Mg/Ag.

This overall device is naturally appropriately (depending on the application) structured, provided with contacts and finally hermetically sealed, since the life of such devices is generally reduced drastically in the presence of water and/or air.

To be able to be used as electroluminescence materials, the oligofluorenes are applied in the form of a film to a substrate, generally by known methods with which those skilled in the art are familiar, e.g. vacuum deposition or from solution by spin coating or using various printing methods (e.g. inkjet printing, offset printing, etc.).

On the other hand, electrophotographic image-forming apparatus have been widely spreading chiefly in offices as printers, copiers, facsimiles, etc. for their capability of high speed processing and high image quality. Broadening of application of the electrophotographic image-forming techniques particularly in the fields of instruments having color output capability and light-duty printers have been of recent interest. Accordingly, electrophotographic image-forming technology realizing high resolution, high gradient and high speed has been being actively researched and developed, and market development of image-forming apparatus reflecting the results of the researches and developments is being undertaken. Coping with such a market trend, improvements along the researches and developments have been added to a photoconductor, which may be said to be the heart of this type of image-forming apparatus, particularly an organic photoconductor (OPC). This kind of organic photoconductor, which is roughly divided into layered or dual-layer, comprises a charge generating layer (CGL) having a charge generation function and a charge transport layer (CTL) having a charge transport function and single-layer ones having a photosensitive layer having both a charge generation function and a charge transport function. The former type of organic photoconductors (OPCs) comprise a cylindrical conductive substrate made of aluminum, etc. on which a CGL and a CTL are stacked in this order. Their application is generally limited to negatively charging image-forming apparatus in nature of the organic material. The latter type construction comprises a cylindrical conductive substrate of aluminum, etc. on which a single-ply photosensitive layer is provided and is mainly applied to positively charging image-forming apparatus which, in principle, easily provide high resolution.

Various layer configurations have been proposed for a photosensitive layer of a positive charging photoconductor, including a layered type having a CGL on a hole-transport layer (HTL) and a single-layer type having a layer containing both a charge generating material (CGM) and a charge transport material (CTM). Positive charging layered type photoconductors are behind negative charging layered type photoconductors in practical application because they have a thin CGL on their surface and therefore involve the problem of poor durability. Positive charging single-layer photoconductors tend to be inferior to negative charging layered photoconductors in electric characteristics such as sensitivity as stated above. This problem is due to because none of the available electron transport materials (ETMs) is equal or superior to available hole-transport materials (HTMs) in mobility.

In recent years, a large number of ETMs and electrophotographic photoconductors using them which deserve attention have been proposed or reported in U.S. Pat. No. 5,328,789, JP-A-4-360148, J. Appl. Phys., vol. 82 (1997), pp. 4957-4961, JP-A-5-92936, Appl. Phys. Lett., vol. 66 (1995), pp. 3433-3435, JP-A-9-151157, Appl. Phys. Lett., vol. 81 (2002), pp. 969-971, Journal of Imaging Science and Technology, vol. 40 (1996), pp. 245-248, J. Appl. Phys., vol. 43 (1972), pp. 5033-5040, Journal of Imaging Science and Technology, vol. 40 (1996), pp. 310-317, and JP-A-10-73937.

Further, single-layer photoreceptors disclosed in U.S. Pat. No. 5,176,976, JP-A-6-130688, U.S. Pat. No. 5,328,788, U.S. Pat. No. 6,743,557, and JP-A-10-239874 have been noted for their high sensitivity, and part of them have been put to practical use.

In the both systems (OLEDs and OPCs), by using a material having a high charge transportability as a charge transporting layer, there is a possibility of obtaining an OLED device with low driving voltage and an electrophotographic photoconductor having a high sensitivity.

In these materials, the charge transporting material is required to quickly transport the carriers in the charge transporting layer.

The transferring velocity of a carrier per unit electric field is called carrier drift mobility. High carrier drift mobility means that the carrier transfers quickly in the charge transporting layer. The carrier drift mobility is specific to the charge transporting material, and hence in order to attain the high carrier drift mobility, it is necessary to use a material showing high carrier drift mobility. The carrier drift mobility by the conventional materials has not yet reached a sufficient level at present.

On the other hand, since the carrier drift mobility depends upon the concentration of the charge transporting material, a method of increasing the concentration of a charge transporting material in a charge transporting layer is employed. The case that the concentration of a charge transporting material becomes the highest is the case that the charge transporting layer is formed by the charge transporting material only and such a charge transporting layer is formed by a vapor deposition method, etc. For example, an organic light emitting diode (OLED) device, etc., is prepared by the method of C. W. Tang and S. A. VanSlyke, Appl. Phys. Lett., 51, 913(1987).

However, with OLED application when the thin layer is a vapor-deposited layer composed of a charge transporting material only, in particular, crystals are liable to deposit and pin holes are liable to form, whereby it is difficult to form the layer having a uniform quality and results in defects on display or even ruin the display.

Also, with OPC application when an organic solvent solution containing a charge transporting material at a high concentration together with a binder polymer is coated to form a coated layer, it is necessary to form a uniform organic thin layer having no deposition of crystals and no formation of pin holes. This is because since a high electric field is applied to the thin layer formed, if the thin layer has fine crystals or pin holes, a dielectric breakdown occurs at the positions of forming the fine crystals or pin holes to cause noise.

As a charge transporting material or a blue emitter, for example, a 9,10-dinaphthyl anthracene derivatives represented by the following formula (II) is proposed in U.S. Pat. No. 5,935,721

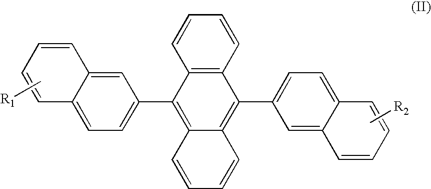

Also, U.S. Pat. No. 5,328,789 discloses a diphenoquinone compound represented by the following formula (III):

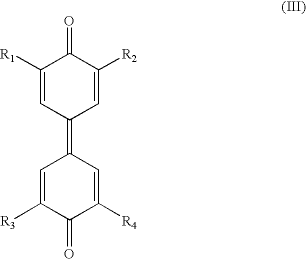

Also, JP-A-3-11355 discloses a distyryl compound represented by the following formula (IV):

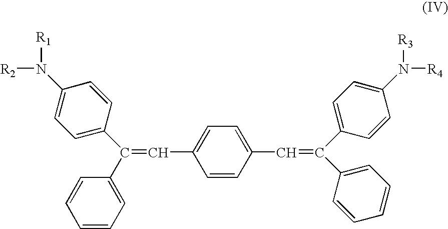

Also, U.S. Pat. No. 4,265,990, U.S. Pat. Nos. 4,273,846 and 5,958,637 disclose a benzidine compound (biphenyl diamine compound) represented by the following formula (V):

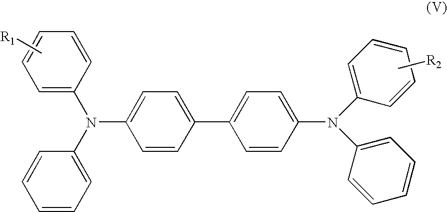

However, in the compounds which were obtained in the Examples or were actually described in the specification, there are problems that the compound is easy to crystallize in thin films to OLED application and to OPC application it is insufficient in the point of solubility in a binder polymer and even when the compound is dissolved in a binder polymer, when a film or layer is formed using it, crystallization occurs, pin holes form, and the film or layer is whitened or becomes brittle, which results in forming defects on the images formed, and hence there is a restriction on the addition amount of the compound.

Apart from the use of the oligofluorenes of the invention in both OLED and OPC devices, these compounds can be used in a very broad range of applications in electronics. Thus, the compounds of the invention can be used in the following devices:

(1) Use in organic solar cells as electron acceptor or electron transport material.

(2) Use in organic ICs as charge transport layer.

(3) Use in further applications, some of which have been mentioned above, e.g. organic solid-state lasers or organic photodetectors.

The invention accordingly provides substituted oligofluorenes of the formula (I) having high carrier drift mobility (electron and hole) and high glass transition temperature (Tg) (hence not tend to crystallize) so as to solve part of the problems mentioned above.

### SUMMARY OF THE PRESENT INVENTION

A main object of the present invention is to provide a new material as a charge transporting material and/or blue emitter, which can show a high carrier drift mobility and is excellent in various characteristics in different applications of the electronics industry.

Another object of the present invention is to provide an OLED device having a layer composed of the charge transporting material of the invention.

Another object of the present invention is to provide an electrophotographic photoconductor having a charge transporting layer containing the charge transporting material of the invention.

As the result of making of various investigations on various compounds, the present inventors have discovered that the object described above can be attained by an oligofluorene derivative represented by the following formula (1) and have succeeded in accomplishing the present invention based on this discovery:

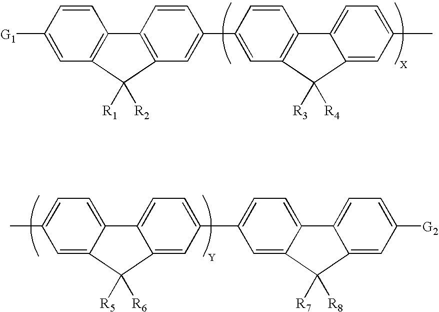

wherein X and Y are an integer of 0 or 1, G₁ and G₂ are independently of either CₙH₂ₙ₊₁ or CₙH₂ₙ₊₁O and n is an integer of 0 to 16, and R₁, R₂, R₃, R₄, R₅, R₆, R₇ and R₈ are independently in each occurrence a hydrogen, C₁₋₂₀ hydrocarby optionally substituted with C₁₋₂₀ alkoxy/aryloxy, thioalkoxy/thioaryloxy, secondary/tertiary amines, hydroxy, carboxylic/sulfonic acids, cyano, and esters; C₆₋₂₀ aryl optionally substituted with C₁₋₂₀ alkoxy/aryloxy, thioalkoxy/thioaryloxy, secondary/tertiary amines, hydroxy, carboxylic/sulfonic acids, cyano, and esters. Each pair of (R₁, R₂), (R₃, R₄), (R₅, R₆) and (R₇, R₈) may also form C₃₋₁₂ cyclic structures with the C-9 carbon of fluorene, said cyclic structures may further contain one or more heteroatoms such as phosphorus, sulfur, oxygen and nitrogen. Preferably R₁, R₂, R₃, R₄, R₅, R₆, R₇ and R₈ are independently in each occurrence a hydrogen, C₁₋₁₂ alkyl optionally substituted with C₁₋₁₂ alkoxy/aryloxy, thioalkoxy/thioaryloxy, secondary/tertiary amines, hydroxy, carboxylic/sulfonic acids, cyano and esters; C₆₋₂₀ aryl optionally substituted with C₁₋₁₂ alkoxy/aryloxy, thioalkoxy/thioaryloxy, secondary/tertiary amines, hydroxy, carboxylic/sulfonic acids, cyano, and esters. Most preferably R₁, R₂, R₃, R₄, R₅, R₆, R₇ and R₈ are independently in each occurrence a hydrogen, C₁₋₈ alkyl optionally substituted with C₁₋₁₀ alkoxy/aryloxy, thioalkoxy/thioaryloxy, secondary/tertiary amines, hydroxy, carboxylic/sulfonic acids, cyano, and esters; C₆₋₁₂ aryl optionally substituted with C₁₋₁₀ alkoxy/aryloxy, thioalkoxy/thioaryloxy, secondary/tertiary amines, hydroxy, carboxylic/sulfonic acids, cyano, and esters.

These and other objectives, features, and advantages of the present invention will become apparent from the following detailed description, the accompanying drawings, and the appended claims.

### BRIEF DESCRIPTION OF THE DRAWINGS

FIG. 1 illustrates the spectrum of the fluorescent emission of the terfluorene 4cc.

FIG. 2 illustrates the spectrum of the ultraviolet light-visible light of the terfluorene 4cc.

FIG. 3 illustrates the spectrum of the fluorescent excitation of the terfluorene 4cc.

FIG. 4 illustrates the physical properties of terfluorene 4aa.

FIG. 5 illustrates the carrier mobility of the terfluorene 4cc under TOC measurement.

FIG. 6 illustrates a structure of OLED.

FIGS. 7A and 7B illustrate the properties of terfluorene when terfluorene is used as a hole transport layer.

FIGS. 8A and 8B illustrate the properties of terfluorene when terfluorene is used as an emission layer.

FIGS. 9A and 9B illustrate the properties of terfluorene when terfluorene is used as an electron transport layer.

FIGS. 10A and 10B illustrate the properties of terfluorene when terfluorene is used as a hole transport layer.

FIGS. 11A and 11B illustrate the properties of terfluorene when terfluorene is used as an electron transport layer.

FIGS. 12A and 12B illustrate the properties of terfluorene when terfluorene is used as an emission layer.

### DETAILED DESCRIPTION OF THE PREFERRED EMBODIMENT

According to a preferred embodiment, a substituted oligofluorene has the following chemical structure.

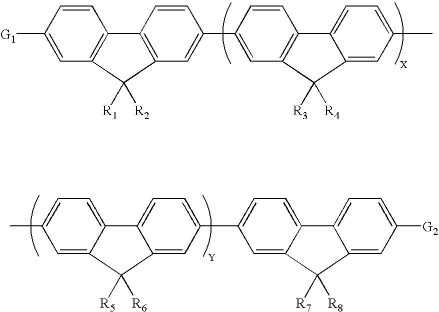

wherein X and Y are an integer of 0 or 1, G₁ and G₂ are independently of either CₙH₂ₙ₊₁ or CₙH₂ₙ₊₁O; and n is an integer of 0 to 16.

R₁, R₂, R₃, R₄, R₅, R₆, R₇ and R₈ are independently in each occurrence a hydrogen. In which, C₁₋₂₀ hydrocarby is optionally substituted with C₁₋₂₀ alkoxy/aryloxy, thioalkoxy/thioaryloxy, secondary/tertiary amines, hydroxy, carboxylic/sulfonic acids, cyano, and esters. C₆₋₂₀ aryl is optionally substituted with C₁₋₂₀ alkoxy/aryloxy, thioalkoxy/thioaryloxy, secondary/tertiary amines, hydroxy, carboxylic/sulfonic acids, cyano, and esters.

Each pair of (R₁, R₂), (R₃, R₄), (R₅, R₆) and (R₇, R₈) may also form C₃₋₁₂ cyclic structures with the C-9 carbon of fluorene, wherein the cyclic structures may further contain one or more heteroatoms such as phosphorus, sulfur, oxygen and nitrogen. Preferably R₁, R₂, R₃, R₄, R₅, R₆, R₇ and R₈ are independently in each occurrence a hydrogen. C₁₋₁₂ alkyl is optionally substituted with C₁₋₁₂ alkoxy/aryloxy, thioalkoxy/thioaryloxy, secondary/tertiary amines, hydroxy, carboxylic/sulfonic acids, cyano and esters. C₆₋₂₀ aryl is optionally substituted with C₁₋₁₂ alkoxy/aryloxy, thioalkoxy/thioaryloxy, secondary/tertiary amines, hydroxy, carboxylic/sulfonic acids, cyano, and esters. The chemical structure shows below in accordance with the R₁, R₂, R₃, R₄, R₅, R₆, R₇ and R₈ as the substituted oligofluorene.

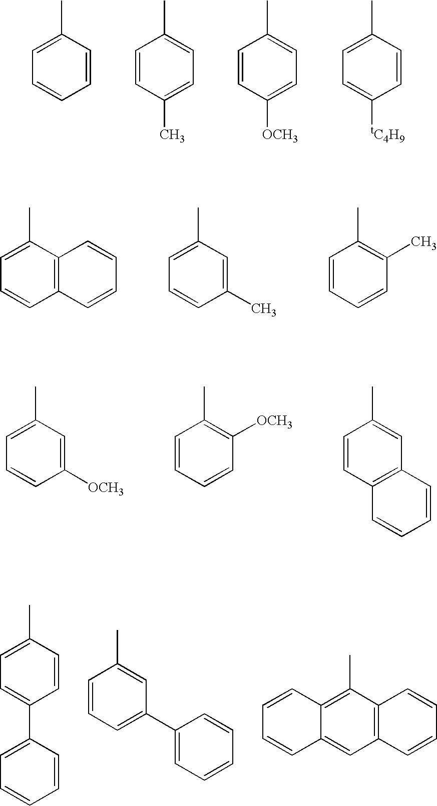

Most preferably R₁, R₂, R₃, R₄, R₅, R₆, R₇ and R₈ are independently in each occurrence a hydrogen. C₁₋₈ alkyl is optionally substituted with C₁₋₁₀ alkoxy/aryloxy, thioalkoxy/thioaryloxy, secondary/tertiary amines, hydroxy, carboxylic/sulfonic acids, cyano, and esters. C₆₋₁₂ aryl is optionally substituted with C₁₋₁₀ alkoxy/aryloxy, thioalkoxy/thioaryloxy, secondary/tertiary amines, hydroxy, carboxylic/sulfonic acids, cyano, and esters. The chemical structure shows below in accordance with the (R₁, R₂), (R₃, R₄), (R₅, R₆) and (R₇, R₈) as the substituted oligofluorene.

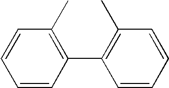

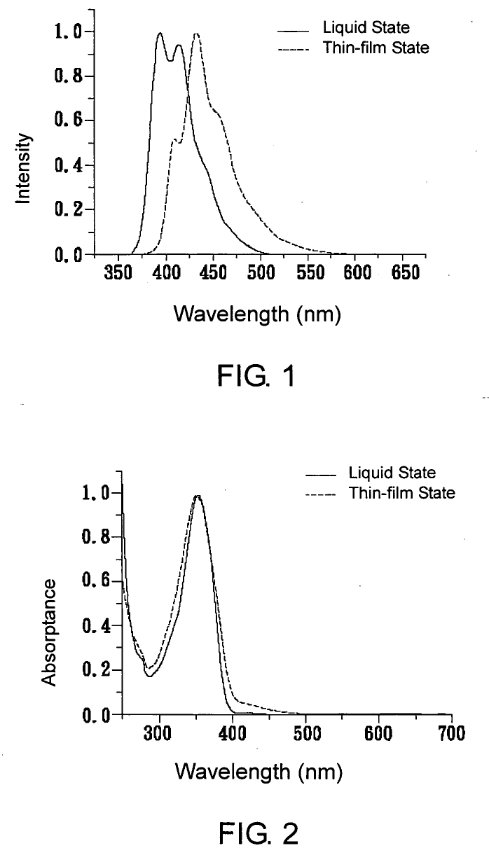

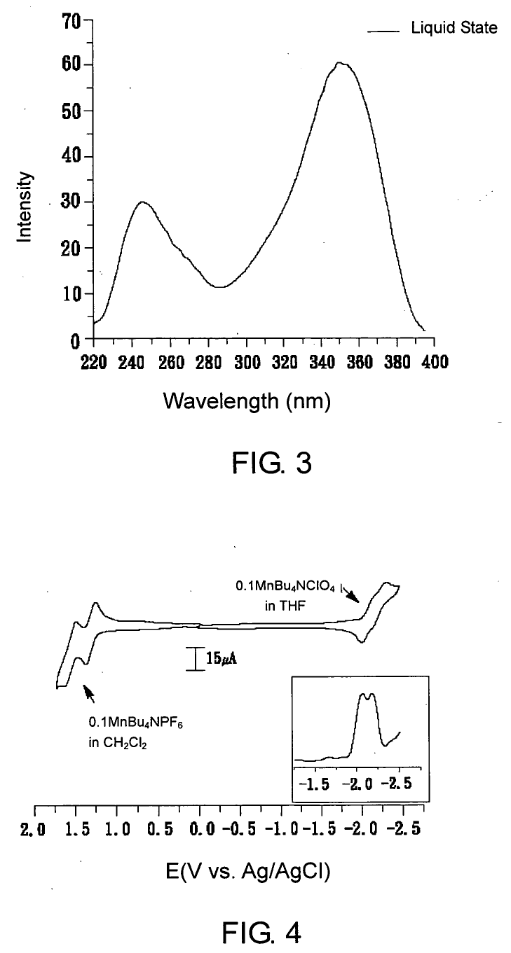

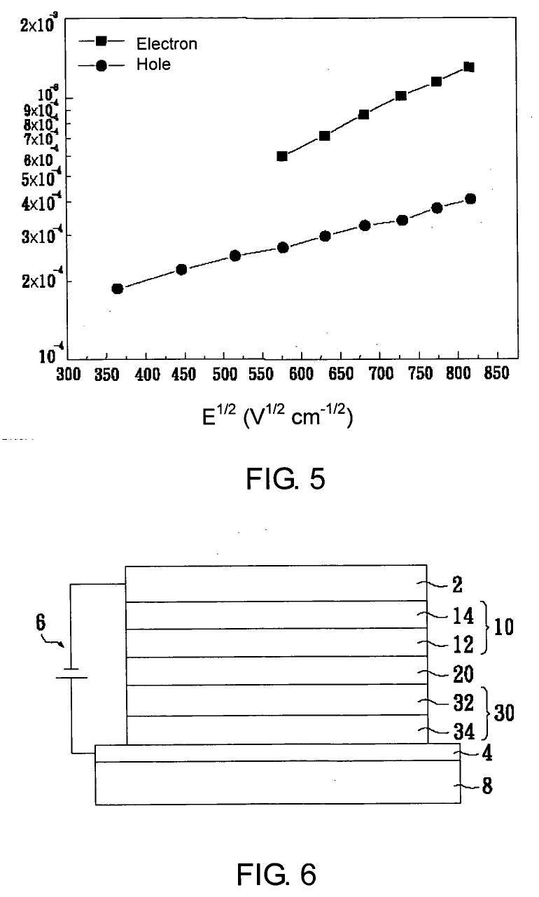

FIGS. 1 to 5 illustrate the physical properties of terfluorene. The following table illustrates the combinations of terfluorene.

| | Ar | Ar | Ar | Ar | G1 = G2 |
|---|---|---|---|---|---|
| 4aa | 2,2′-biphernyl | | 2,2′-biphernyl | | H |
| 4bb | Ph | Ph | Ph | Ph | H |
| 4cc | p-Tol | p-Tol | p-Tol | p-Tol | H |

| | | | | |
|---|---|---|---|---|
| 4ab | 2,2′-biphernyl | Ph | Ph | H |
| 4ac | 2,2′-biphernyl | p-Tol | p-Tol | H |
| 4ad | 2,2′-biphernyl | p-Tol | 1-Np | H |

As shown in FIG. 6, an organic Light Emitting Diode (OLED) comprises an electron-transport layer 10, an organic emission layer 20 and a hole-transport layer 30, wherein the electron-transport layer 10 and the hole-transport layer 30 are electrically overlapped between a cathode 2 and a transparent anode (indium-tin oxide ITO) 4 through an electric circuit 6. When an external electric field is applied to the organic LED, electrons are injected from cathode and holes injected from anode into the corresponding transporting layers, where they travel in the applied filed until they meet and form a luminescent excited state in the emission layer. Therefore, the electrons generate spectrum when the electrons return to the ground state from the excited state.

Accordingly, the electron-transport layer 10 comprises an electron transport layer 12 and an electron injection layer 14. The hole-transport layer 30 comprises a hole transport layer 32 and a hole injection layer 34, wherein the electron injection layer 14 and the hole injection layer 34 are adapted for enhancing the conductivity between the cathode 2 and the electron transport layer 12 and the conductivity between the transparent anode 4 and the hole transport layer 32, so as to enhance the electron injection from the electron-transport layer 10 to the hole-transport layer 30.

Since the substituted oligofluorenes of the present invention has high carrier drift mobility (electron and hole), the substituted oligofluorene can be incorporated with the OLED to simplify the structural configuration thereof.

An application of the terfluorene illustrates the use of substituted oligofluorene for the multilayer OLED, wherein terfluorene 4aa (T-Spiro) and terfluorene 4cc (T-Tol) are shown as follow.

(1) Terfluorene 4aa (T-Spiro): glass substrate/ITO/PEDT:PSS (30 nm)/Terfluorene compound 4aa (50 nm)/TPBI (37 nm)/LiF (0.5 nm)/Al (150 nm);

(2) Terfluorene 4 cc (T-Tol): glass substrate/ITO/PEDT:PSS (30 nm)/Terfluorene compound 4 cc (50 nm)/TPBI (37 nm)/LiF (0.5 nm)/Al (150 nm).

The transparent anode 4 is initially coated on the glass substrate wherein the inorganic and organic layers are then overlapped thereon. The cathode which is made of Al is coated on the glass substrate, wherein the high conductive element of polyethylene dioxythiophene/polystyrene sulphonate (PEDT:PSS) forms the hole injection layer 34, tris-(8-hydroxyqulinoline) aluminum (Alq₃) forms the electron transport layer 12, LiF forms the electron injection layer 14, terfluorene forms the hole transport layer 32.

Accordingly, the chemical structure of terfluorene 4aa (T-Spiro) is:

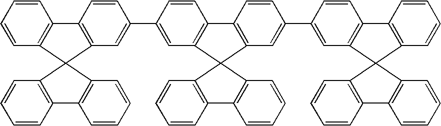

The chemical structure of terfluorene 4 cc (T-Tol) is:

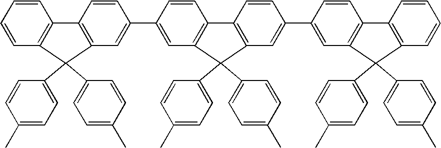

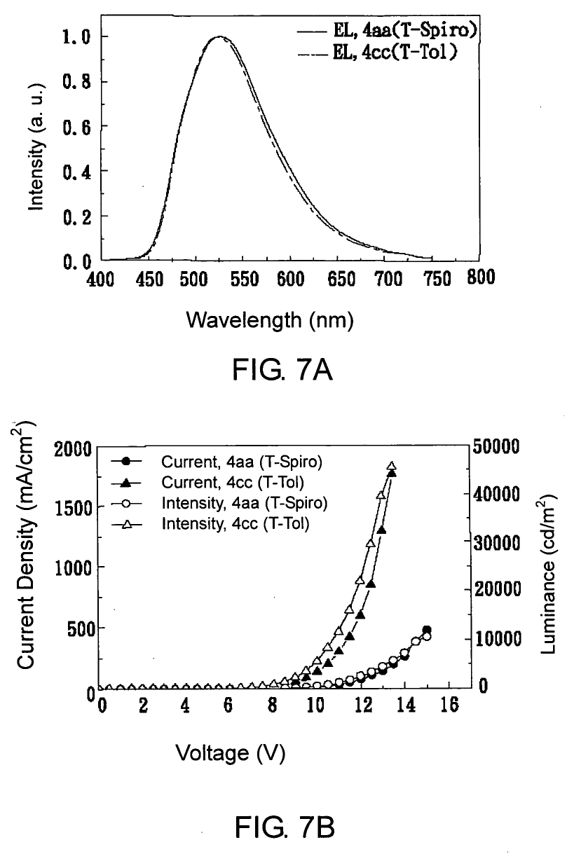

As shown in FIGS. 7A and 7B, when the terfluorene 4aa and the terfluorene 4cc form as the hole transport layer 32 to enhance the current through the OLED, the OLED generates a relatively high intensity spectrum (terfluorene 4aa>10000 cd/m², terfluorene 4cc>40000 cd/m²) with 1.2-1.4% high external electroluminescence quantum efficiency.

Terfluorene can be used as the emission layer 20 for the multilayer OLED. Accordingly, terfluorene 4cc is used with glass substrate/ITO/PEDT:PSS (30 nm)/TCTA (40 nm)/Terfluorene compound 4cc (30 nm)/TPBI (30 nm)/LiF (0.5 nm)/Al (150 nm).

The transparent anode 4 is initially coated on the glass substrate wherein the inorganic and organic layers are then overlapped thereon. The cathode which is made of Al is coated on the glass substrate, wherein the high conductive element of polyethylene dioxythiophene/polystyrene sulphonate (PEDT:PSS) forms the hole injection layer 34, 4,4′,4″-Tris-(carbazol-9-yl)-triphenylamine (TCTA) forms the hole transport layer 32, 1,3,5-tris(N-phenylbenzimidazol-2-yl) benzene (TPBI) form the electron transport layer 12, LiF forms the electron injection layer 14, and terfluorene 4cc forms the emission layer 20.

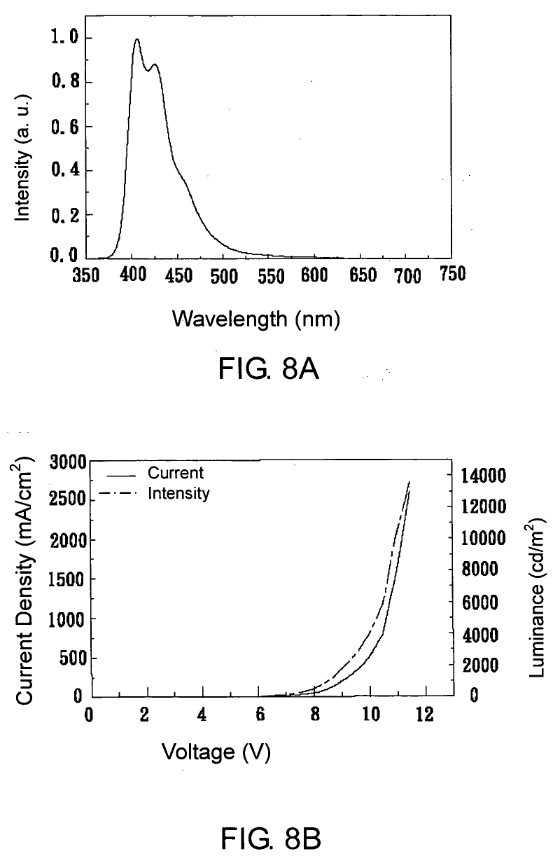

As shown in FIGS. 8A and 8B, the terfluorene 4cc forms as the blue emitter to generate a relatively high intensity spectrum (>130000 cd/m²) with ˜3.8% high external electroluminescence quantum efficiency.

Terfluorene can be used as the electron transport layer 12 for the multilayer OLED. Accordingly, terfluorene 4aa (T-Spiro) and terfluorene 4cc (T-Tol) are used with the following components:

(1) glass substrate/ITO/PEDT:PSS (30 nm)/α-NPD (20 nm)/Terfluorene compound 4cc (60 nm)/Alq3 (20 nm)/LiF (0.5 nm)/Al (150 nm);

(2) glass substrate/ITO/PEDT:PSS (30 nm)/α-NPD (40 nm)/Terfluorene compound 4 cc (40 nm)/Alq3 (20 nm)/LiF (0.5 nm)/Al (150 nm).

The transparent anode 4 is initially coated on the glass substrate wherein the inorganic and organic layers are then overlapped thereon. The cathode which is made of Al is coated on the glass substrate, wherein the high conductive element of polyethylene dioxythiophene/polystyrene sulphonate (PEDT:PSS) forms the hole injection layer 34, α-naphthylphenylbiphenyl diamine (α-NPD) forms the hole transport layer 32, tris-(8-hydroxyquinolinc) aluminum (Alq₃) form the electron transport layer 12, and LiF forms the electron injection layer 14, wherein the electron transport layer 12 is made of terfluorene 4cc.

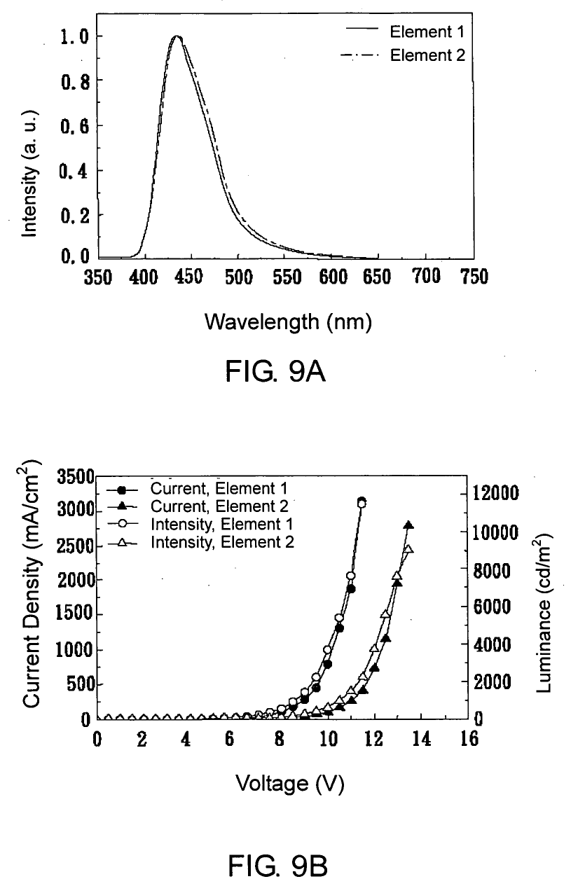

As shown in FIGS. 9A and 9B, when the terfluorene 4cc forms as the electron transport layer 12 to enhance the current through the OLED, the OLED generates a relatively high intensity spectrum (terfluorene>10000 cd/m²) with 1% high external electroluminescence quantum efficiency.

Terfluorene can be used as the hole transport-emission layer for the multilayer OLED. Accordingly, terfluorene 4aa (T-Spiro) and terfluorene 4cc (T-Tol) are used with the following components:

(1) Terfluorene 4aa (T-Spiro): glass substrate/ITO/PEDT:PSS (30 nm)/Terfluorene compound 4aa (50 nm)/TPBI (37 nm)/LiF (0.5 nm)/Al (150 nm);

(2) Terfluorene 4cc (T-Tol): glass substrate/ITO/PEDT:PSS (30 nm)/Terfluorene compound 4cc (50 nm)/TPBI (37 nm)/LiF (0.5 nm)/Al (150 nm).

The transparent anode 4 is initially coated on the glass substrate wherein the inorganic and organic layers are then overlapped thereon. The cathode which is made of Al is coated on the glass substrate, wherein the high conductive element of polyethylene dioxythiophene/polystyrene sulphonate (PEDT:PSS) forms the hole injection layer 34, 1,3,5-tris(N-phenylbenzimidazol-2-yl)benzene (TPBI) forms the electron transport layer 12, LiF forms the electron injection layer 14, and terfluorene forms the hole transport-emission layer which is formed by integrally combining the hole transport layer 32 and the emission layer 20.

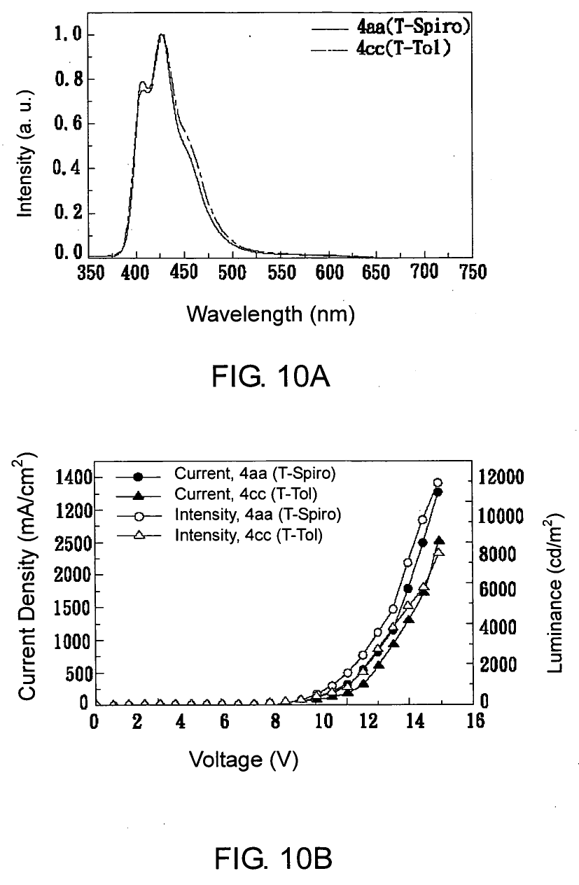

As shown in FIGS. 10A and 10B, the terfluorene forms as the blue emitter to generate a relatively high intensity spectrum (>4000-5000 cd/m²) with ˜2.5-3% high external electroluminescence quantum efficiency.

Terfluorene can be used as the electron transport-emission layer for the multilayer OLED. Accordingly, terfluorene 4cc is used with glass substrate/ITO/PEDT:PSS (30 nm)/TCTA (50 nm)/Terfluorene compound 4 cc (50 nm)/LIF (0.5 nm)/Al (150 nm).

The transparent anode 4 is initially coated on the glass substrate wherein the inorganic and organic layers are then overlapped thereon. The cathode which is made of Al is coated on the glass substrate, wherein the high conductive element of polyethylene dioxythiophene/polystyrene sulphonate (PEDT:PSS) forms the hole injection layer 34, 4,4′,4″-Tris-(carbazol-9-yl)-triphenylamine forms the hole transport layer 32, LiF forms the electron injection layer 14, and terfluorene 4cc forms the electron transport-emission layer which is formed by integrally combining the electron transport layer 12 with the emission layer 20.

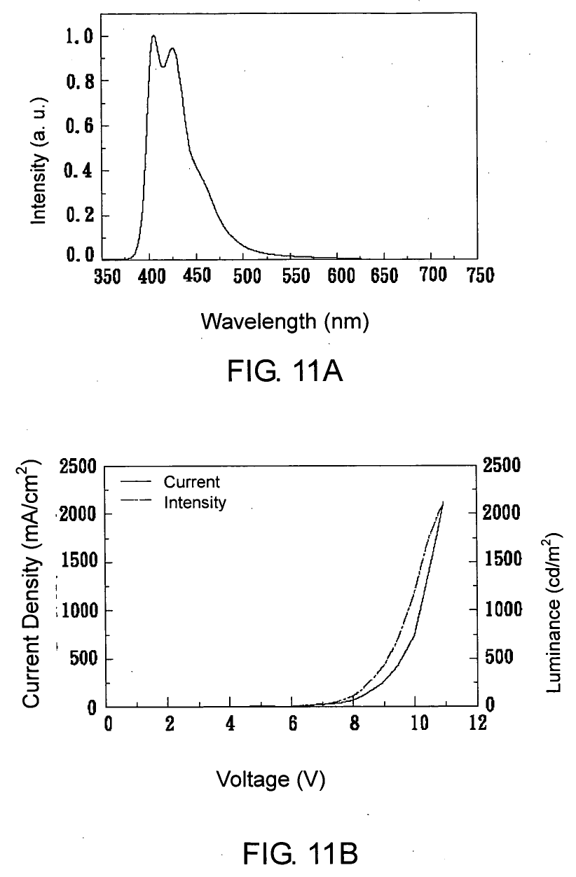

As shown in FIGS. 11A and 11B, the terfluorene 4cc forms as the blue emitter to generate a relatively high intensity spectrum (>2000 cd/m²) with ˜0.6% high external electroluminescence quantum efficiency.

Terfluorene can be used as the electron transport layer 12, the emission layer 20, and the hole transport layer 32 for the single-layer OLED.

The transparent anode 4 is initially coated on the glass substrate wherein the inorganic and organic layers are then overlapped thereon. The cathode which is made of Al is coated on the glass substrate, wherein the high conductive element of polyethylene dioxythiophene/polystyrene sulphonate (PEDT:PSS) forms the hole injection layer 34, LiF forms the electron injection layer 14, and terfluorene forms the emission layer 20. Accordingly, the terfluorene can also forms the electron transport layer 12 and/or the hole transport layer 32.

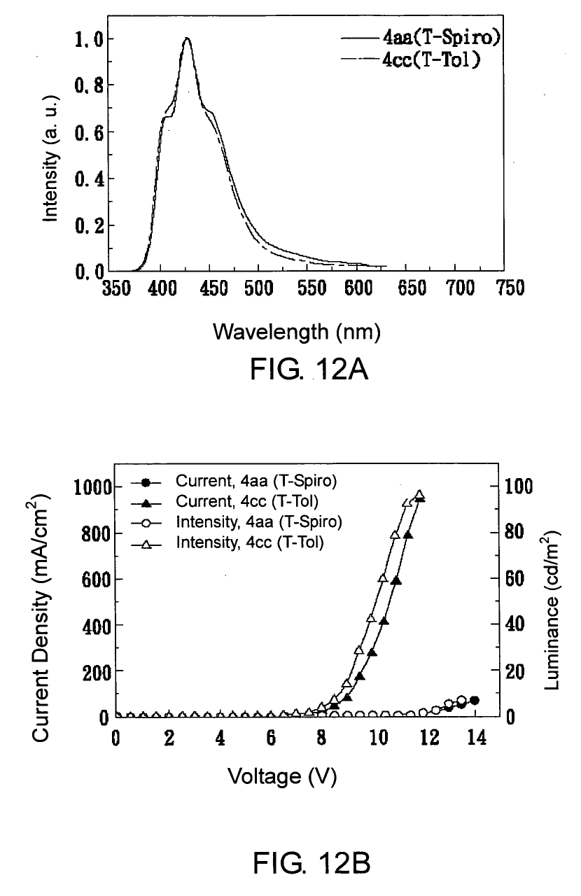

As shown in FIGS. 12A and 12B, the terfluorene forms as the blue emitter to generate a relatively high intensity spectrum for the single-layer OLED.

According to the preferred embodiment, the substituted oligofluorene can be incorporated with an OPC (organic photoconductor). Accordingly, the OPC comprises a composition of a charging generating material and a charge transporting material, wherein the charge transporting material contains a substituted oligofluorene. The substituted oligofluorene can be terfluorene compound 4aa, terfluorene compound 4bb, terfluorene compound 4cc, terfluorene compound 4ab, terfluorene compound 4ac, or terfluorene compound 4ad.

Accordingly, the OPC can be a duel layer OPC having a charging generating layer made of charging generating material and a charge transporting layer which is made of charge transporting material and is overlapped on the charging generating layer. Alternatively, the OPC can be a single layer OPC that both the charging generating material and the charge transporting material are formed in one single layer.

The molecular structures of illustrative compounds 4aa, 4bb, 4cc, 4ab, 4ac, 4ad and 5 and comparative compounds 6, 7 and 8 appeared in following examples and comparative examples are shown below and the related literatures are cited as references here.

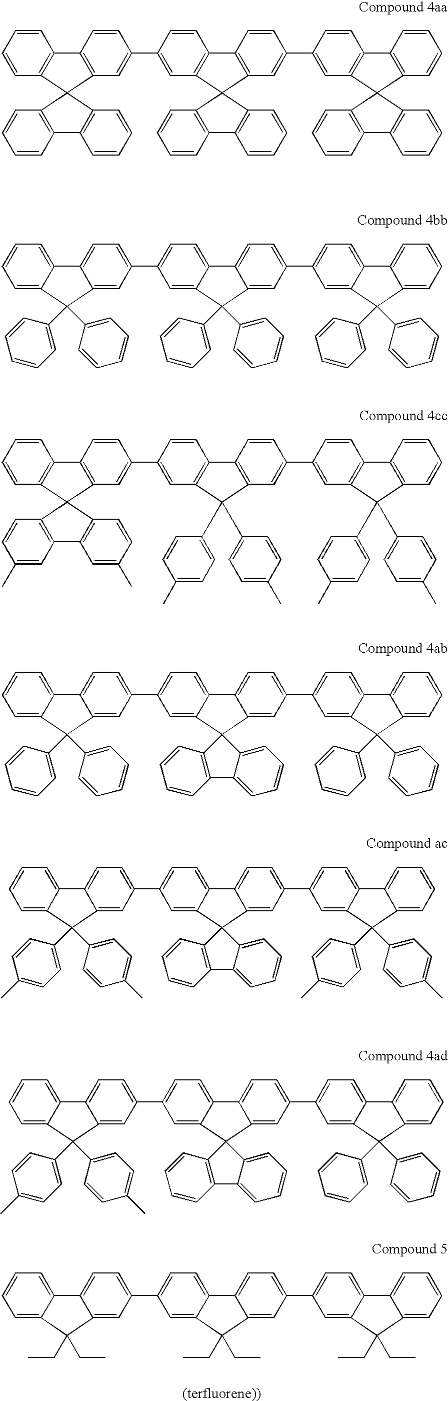

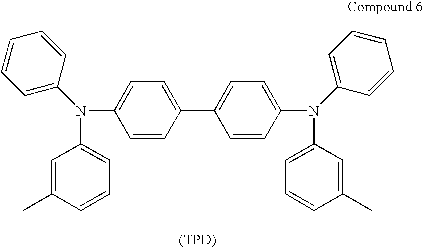

Compound 6, a biphenyl diamine derivative named TPD, is a conventional hole transporting material used for OLED and electrophotographic photoconductor and can be founded in U.S. Pat. No. 4,588,666, U.S. Pat. No. 6,444,333, U.S. Pat. No. 6,476,265, U.S. Pat. No. 5,958,637, U.S. Pat. No. 5,935,721, U.S. Pat. No. 4,877,702, U.S. Pat. No. 4,265,990 and U.S. Pat. No. 4,273,846.

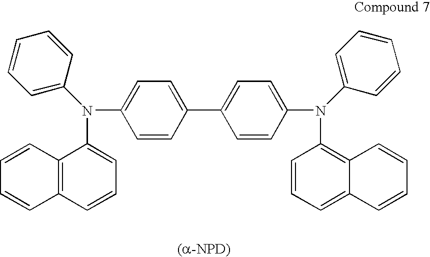

Compound 7, a biphenyl diamine derivative named α-NPD, is a conventional hole transporting material used for OLED and electrophotographic photoconductor and can be founded in U.S. Pat. No. 4,588,666, U.S. Pat. No. 6,444,333, U.S. Pat. No. 6,476,265, U.S. Pat. No. 5,958,637, U.S. Pat. No. 5,935,721, U.S. Pat. No. 4,877,702, U.S. Pat. No. 4,265,990 and U.S. Pat. No. 4,273,846.

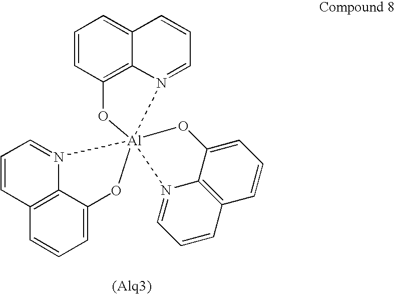

Compound 8, tris(8-hydroxyquinoline)aluminum (Alq3), is a conventional electron transporting material used for OLED and can be founded in U.S. Pat. No. 5,998,046, Adv. Funct. Mater. 2003, 13, pp 108 and Mater. Sci. & Engineering 2002, 39, 143.

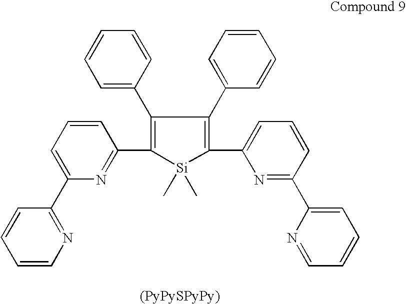

Compound 9, a silole derivative named PyPySPyPy, is a conventional electron transporting material used for OLED and can be founded in U.S. Pat. No. 6,376,694 and Chem. Phys. Lett. 2001, 339, 161.

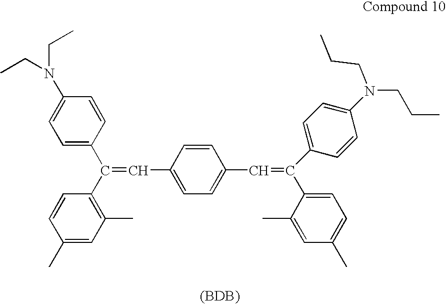

Compound 10, a 1,4-Bis(2′,2′-diphenylvinyl)benzene derivative named BDB, is a conventional charge transporting material used for electrophotographic photoconductor and can be founded in U.S. Pat. No. 5,750,785, U.S. Pat. No. 5,856,013 and U.S. Pat. No. 5,521,042

Each of illustrative compounds 4aa, 4bb, 4cc, 4ab, 4ac, 4ad and 5 and comparative compounds 6, 7, 8, 9 and 10 described above was subjected to a time-of-flight measurement (TOF) for determining the carrier drift mobility. The results obtained at E=1×10⁵ V/cm are shown in table below.

| | Compound | | | | | | |
|---|---|---|---|---|---|---|---|
| | 4ab | 5 | 6 | 7 | 8 | 9 | 10 |
| Carrier polarity | electron | electron | hole | hole | electron | electron | electron |
| | hole | hole | | | | | |
| Drift mobility at | 1.4 × 10⁻³ | 1.8 × 10⁻³ | 8.2 × 10⁻⁴ | 6.2 × 10⁻⁴ | 1.4 × 10⁻⁷ | 1.6 × 10⁻⁵ | 6.3 × 10⁻⁵ |
| E = 1 × 10⁵ V/cm | | | | | | | |
| ((cm⁻²/Vs)) | 4.3 × 10⁻³ | 9.5 × 10⁻⁴ | | | | | |

Each of illustrative compounds 4aa, 4bb, 4cc, 4ab, 4ac and 4ad described above was subjected to a thermal analysis using a differential scanning calorimeter (DSC) for determining the glass transition temperature (Tg), the crystallization temperature (Tc) and the melting point (Tm). That is, when the temperature of each of the foregoing compounds was increased at a rate of 10° C./minute, an endothermic peak by melting of the compound was observed. The isotropic liquid obtained by melting in this case was quickly cooled by liquid nitrogen to form a transparent glassy compound. Then, when the temperature of the glassy compound was increased again at a rate of 10° C./minute, a glass transition point was seen and the temperature at the case was defined as the glass transition temperature (Tg) of the compound. Thereafter, when the temperature was further increased, an exothermic peak by crystallization was observed and the temperature at the case was defined as the crystallization temperature (Tc) of the compound. When the temperature was further increased, an endothermic peak by melting was observed and the temperature at the case was defined as the melting point (Tm) of the compound.

The thin-film samples of the charge transporting layer for OPC characterization were prepared by mixing and dissolving 1 part by weight of each oligofluorenes and 1 part by weight of a polycarbonate resin in 8 parts by weight of tetrahydrofurane, each solution thus obtained was coated on a sheet formed by vapor-depositing silver (Ag) on a glass substrate by a doctor blade and dried for 3 hours at 80° C. to form a charge transporting layer.

Furthermore, an aluminum thin layer was vapor-deposited on each of the charge transporting layers and the carrier drift mobilities were measured.

Charge transport in films was typically characterized in a vacuum by applying the time-of-flight (TOF) transient photocurrent technique on glass/Ag (30 nm)/organic (1.5 μm)/Al (150 nm) samples. Pulsed illumination (third harmonic of Nd:YAG laser, 355 nm, 10 ns) through the semitransparent electrode (Ag) induces photogeneration of a thin sheet of excess carriers. The sample thickness had been chosen to be much larger than the optical absorption depth of the excitation. Depending on the polarity of the applied bias, selected photogenerated carriers (holes or electrons) are swept across the sample with a transit time Tₜ. With the applied bias V and the sample thickness D, the applied electric field E is then V/D, and the carrier mobility is given by μ=D/(Tₜ·E)=D2/(V·T₁).

The results obtained at E 8.1×10⁵ V/cm are shown in Table 4 and 5 below.

TABLE 4

| Compound | Hole Drift Mobility (cm²/Vs) |
|---|---|
| 5(terfluorene) | 5.6 × 10⁻⁵ |
| 6(TPD) | 9.0 × 10⁻⁶ |

TABLE 5

| Compound | Electron Drift Mobility (cm²/Vs) |
|---|---|
| 5(terfluorene) | 2.0 × 10⁻⁴ |
| 10(BDB) | ^{a}1.5 × 10⁻⁵ |

^{a}the data obtained from U.S. Pat. No. 5750785

From the results shown above, it can be seen that usually if the carrier drift mobility of a charge transporting layer is about 8 to 9×10⁻⁶ (cm.²/Vs), the charge transporting layer is considered to be excellent, while by using the compounds of the present invention, a higher carrier drift mobility is obtained.

In the case of dissolving illustrative Compound 5 in a polycarbonate resin as a binder, the compound was added to a polycarbonate resin in a weight ratio of 40%, 50%, 66%, or 80% to the whole mixture and was mixed therewith and dissolved therein with tetrahydrofuran of 4 times (by weight) the amount of the solid components. In these cases, the compound was uniformly dissolved and the solubility of the charge transporting material was high.

Each solution was coated on a sheet formed by vapor-depositing silver on a glass substrate by the same manner as in previous Application Examples and the mixture prepared above was dried for 3 hours at 80° C. to form each charge transporting layer. When the charge transporting layer containing the charge transporting material at a high concentration as described above, the uniform layer having neither deposition of crystals nor the formation of pinholes could be formed. Then, an aluminum thin layer was vapor-deposited on each of the charge transporting layers and the carrier mobility at E=8.1×10⁵ V/cm was measured. The results are shown in table 6 and 7 below.

Furthermore, 100% illustrative compound 5 without using a binder was mixed with and dissolved in tetrahydrofuran in an amount of twice (by weight) the amount of the solid material and the solution was added dropwise onto the foregoing sheet by a Pasteur pipet. After drying at room temperature, the layer was further dried at 80° C. for 3 hours under reduced pressure to form a charge transporting layer.

As described above, when the charge transporting layer composed of the charge transporting material only without the addition of a binder was formed, a uniform layer having neither the deposition of crystals nor the formation of pinholes could be formed.

The carrier drift mobility at E=8.1×10⁵ V/cm was measured by the same manner as above and the result is shown in Table 6 and 7 below.

TABLE 6
(Mobility Change to Concentration of Illustrative Compound 5)

| Weight % | Hole Drift Mobility (cm²/Vs) |
|---|---|
| 40 | 9.0 × 10⁻⁶ |
| 50 | 5.6 × 10⁻⁵ |
| 66 | 2.3 × 10⁻⁴ |
| 100 | ^{a}9.5 × 10⁻⁴ |

^{a}The carrier drift mobility at E = 1.0 × 10⁵ V/cm

TABLE 7
(Mobility Change to Concentration of Illustrative Compound 5)

| Weight % | Electron Drift Mobility (cm²/Vs) |
|---|---|
| 40 | 5.4 × 10⁻⁵ |
| 50 | 2.3 × 10⁻⁴ |
| 66 | 9.3 × 10⁻⁴ |
| 100 | ^{a}1.8 × 10⁻³ |

^{a}The carrier drift mobility at E = 1.0 × 10⁵ V/cm

From the results shown above, it can be seen that in the case of using the compound of the present invention at 100%, the high hole mobility of 9.5×10⁻⁴ and the electron mobility of 1.4×10⁻³ cm²/Vs can be obtained and thus the compound of the present invention is very useful.

In the case of dissolving Comparative Compound 6 in a polycarbonate resin as a binder, the compound was added such that the weight ratio of Comparative Compound 1 became 40%, 50%, or 66% of the whole mixture and mixed and dissolved with tetrahydrofuran of 4 times (by weight) the amount of the solid components.

Each of the solutions obtained was coated on a sheet formed by vapor-depositing silver on a glass substrate by the same manner as previous Application Examples and dissolved products described above was used as the charge transporting material followed by drying at 80° C. for 3 hours.

The results showed that when the weight ratio of Comparative Compound was 40% and 50%, uniform coated layers were obtained but when the weight ratio thereof was 66%, crystals deposited on the whole surface of the coated layer.

As described above, the oligofluorene of the present invention has a good solubility and when a film or layer containing the compound at a high concentration is formed by increasing the addition amount of the compound, a uniform and stabilized film or layer having an excellent faculty as a charge transporting material can be formed. Thus, when an electrophotographic photoreceptor is prepared using the compound of the present invention for the charge transporting layer, the charge transporting layer can show a high carrier drift mobility and the electrophotographic photoreceptor has good characteristics of a high sensitivity and giving no residual potential. Thus, the oligofluorene of the present invention is an industrially excellent compound.

One skilled in the art will understand that the embodiment of the present invention as shown in the drawings and described above is exemplary only and not intended to be limiting.

It will thus be seen that the objects of the present invention have been fully and effectively accomplished. It embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A substituted oligofluorene, which has a chemical structure of: 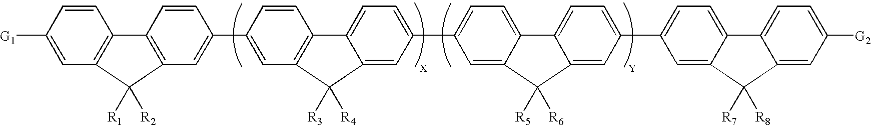
wherein X and Y are an integer of 0 or 1, G₁ and G₂ are independently of either CₙH₂ₙ₊₁ or CₙH₂ₙ₊₁O; and n is an integer of 0 to 16.

2. The substituted oligofluorene, as recited in claim 1, wherein said R₁, R₂, R₃, R₄, R₅, R₆, R₇ and R₈ are selected from the group consisting of the following chemical structures:

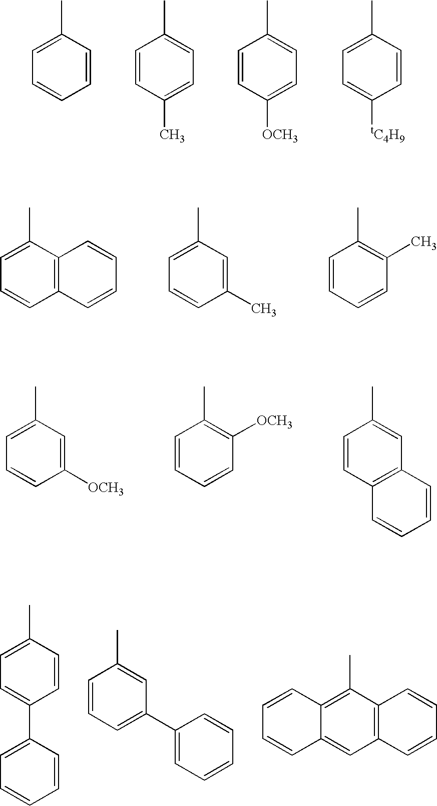

3. The substituted oligofluorene, as recited in claim 1, wherein said (R₁, R₂), (R₃, R₄), (R₅, R₆) and (R₇, R₈) R₈ are selected from the group consisting of the following chemical structure:

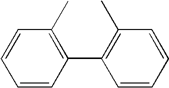

4. An OLED (organic Light Emitting Diode), comprising a substrate, a hole-transport layer, an organic emission layer, an electron-transport layer overlapped with each other, wherein said electron-transport layer comprises an electron injection layer connecting to a cathode and an electron transport layer coupling with said emission layer, wherein said hole-transport layer comprises a hole injection layer connecting to an anode and a hole transport layer coupling with said emission layer, wherein said OLED contains a substituted oligofluorene having a chemical structure of: 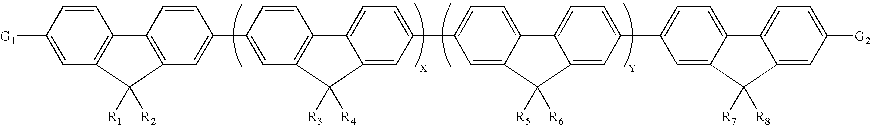
wherein X and Y are an integer of 0 or 1, G₁ and G₂ are independently of either CₙH₂ₙ₊₁ or CₙH₂ₙ₊₁O; and n is an integer of 0 to 16.

5. The OLED, as recited in claim 4, wherein C₁₋₂₀ hydrocarby is optionally substituted with C₁₋₂₀ alkoxy/aryloxy, thioalkoxy/thioaryloxy, secondary/tertiary amines, hydroxy, carboxylic/sulfonic acids, cyano, and esters.

6. The OLED, as recited in claim 4, wherein C₆₋₂₀ aryl is optionally substituted with C₁₋₂₀ alkoxy/aryloxy, thioalkoxy/thioaryloxy, secondary/tertiary amines, hydroxy, carboxylic/sulfonic acids, cyano, and esters.

7. The OLED, as recited in claim 5, wherein C₆₋₂₀ aryl is optionally substituted with C₁₋₂₀ alkoxy/aryloxy, thioalkoxy/thioaryloxy, secondary/tertiary amines, hydroxy, carboxylic/sulfonic acids, cyano, and esters.

8. The OLED, as recited in claim 4, wherein said hole transport layer contains said substituted oligofluorene.

9. The OLED, as recited in claim 4, wherein said emission layer contains said substituted oligofluorene.

10. The OLED, as recited in claim 4, wherein said electron transport layer contains said substituted oligofluorene.

11. The OLED, as recited in claim 4, wherein said hole transport layer is integral with said emission layer to form a hole transport-emission layer, wherein said hole transport-emission layer contains said substituted oligofluorene.

12. The OLED, as recited in claim 4, wherein said electron transport layer is integral with said emission layer to form an electron transport-emission layer, wherein said electron transport-emission layer contains said substituted oligofluorene.

13. An OLED (organic Light Emitting Diode), comprising a substrate, a hole-transport layer, an organic emission layer, an electron-transport layer overlapped with each other, wherein said electron-transport layer comprises an electron injection layer connecting to a cathode and an electron transport layer coupling with said emission layer, wherein said hole-transport layer comprises a hole injection layer connecting to an anode and a hole transport layer coupling with said emission layer, wherein said OLED contains a substituted oligofluorene which is a terfluorene compound.

14. The OLED, as recited in claim 13, wherein said terfluorene compoung is selected from a group consisting of:
a first terfluorene compound having a chemical structure of 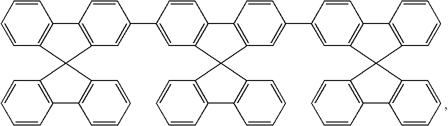
a second terfluorene compound having a chemical structure of 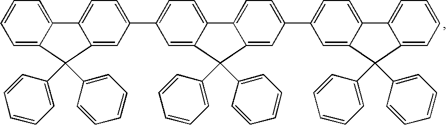
a third terfluorene compound having a chemical structure of 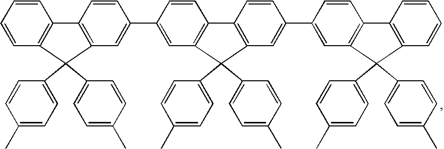
a fourth terfluorene compound having a chemical structure of 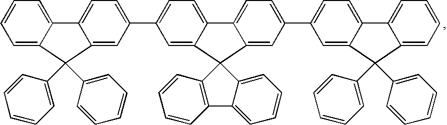
a fifth terfluorene compound having a chemical structure of 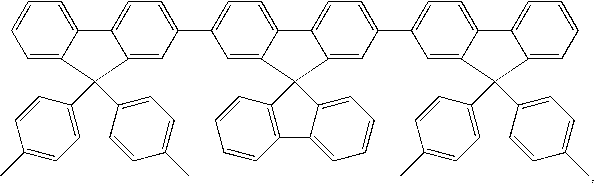
, and
a sixth terfluorene compound having a chemical structure of

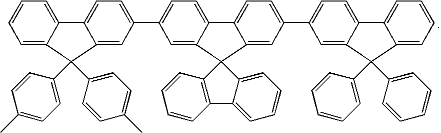

15. The OLED, as recited in claim 13, wherein said hole transport layer contains said substituted oligofluorene.

16. The OLED, as recited in claim 14, wherein said hole transport layer contains said substituted oligofluorene.

17. The OLED, as recited in claim 13, wherein said emission layer contains said substituted oligofluorene.

18. The OLED, as recited in claim 14, wherein said emission layer contains said substituted oligofluorene.

19. The OLED, as recited in claim 13, wherein said electron transport layer contains said substituted oligofluorene.

20. The OLED, as recited in claim 14, wherein said electron transport layer contains said substituted oligofluorene.

21. The OLED, as recited in claim 13, wherein said hole transport layer is integral with said emission layer to form a hole transport-emission layer, wherein said hole transport-emission layer contains said substituted oligofluorene.

22. The OLED, as recited in claim 14, wherein said hole transport layer is integral with said emission layer to form a hole transport-emission layer, wherein said hole transport-emission layer contains said substituted oligofluorene.

23. The OLED, as recited in claim 13, wherein said electron transport layer is integral with said emission layer to form an electron transport-emission layer, wherein said electron transport-emission layer contains said substituted oligofluorene.

24. The OLED, as recited in claim 14, wherein said electron transport layer is integral with said emission layer to form an electron transport-emission layer, wherein said electron transport-emission layer contains said substituted oligofluorene.

25. An OPC (organic photoconductor), comprising a composition of a charging generating material and a charge transporting material, wherein said charge transporting material contains a substituted oligofluorene having a chemical structure of: 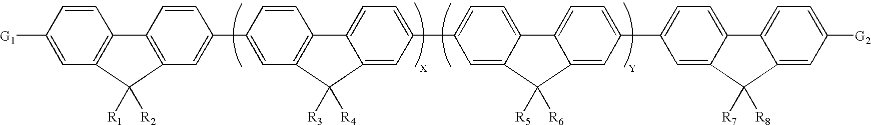
wherein X and Y are an integer of 0 or 1, G₁ and G₂ are independently of either CₙH₂ₙ₊₁ or CₙH₂ₙ₊₁O; and n is an integer of 0 to 16.

26. The OPC, as recited in claim 25, wherein said R₁, R₂, R₃, R₄, R₅, R₆, R₇ and R₈ are independently in each occurrence a hydrocarby.

27. The OPC, as recited in claim 26, wherein C₁₋₂₀ hydrocarby is optionally substituted with C₁₋₂₀ alkoxy/aryloxy, thioalkoxy/thioaryloxy, secondary/tertiary amines, hydroxy, carboxylic/sulfonic acids, cyano, and esters.

28. The OPC, as recited in claim 26, wherein C₆₋₂₀ aryl is optionally substituted with C₁₋₂₀ alkoxy/aryloxy, thioalkoxy/thioaryloxy, secondary/tertiary amines, hydroxy, carboxylic/sulfonic acids, cyano, and esters.

29. The OPC, as recited in claim 27, wherein C₆₋₂₀ aryl is optionally substituted with C₁₋₂₀ alkoxy/aryloxy, thioalkoxy/thioaryloxy, secondary/tertiary amines, hydroxy, carboxylic/sulfonic acids, cyano, and esters.

30. An OPC (organic photoconductor), comprising a composition of a charging generating material and a charge transporting material, wherein said charge transporting material contains a substituted oligofluorene which is a terfluorene compound.

31. The OPC, as recited in claim 30, wherein said terfluorene is selected from a group consisting of:
a first terfluorene compound having a chemical structure of 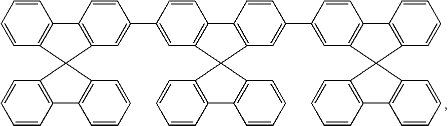
a second terfluorene compound having a chemical structure of 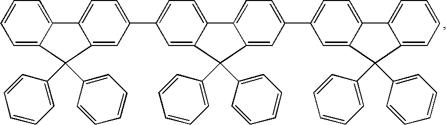
a third terfluorene compound having a chemical structure of 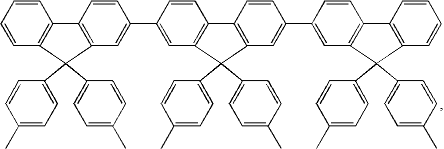
a fourth terfluorene compound having a chemical structure of 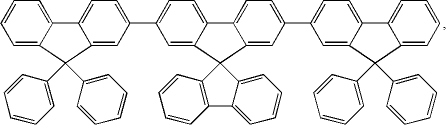
a fifth terfluorene compound having a chemical structure of 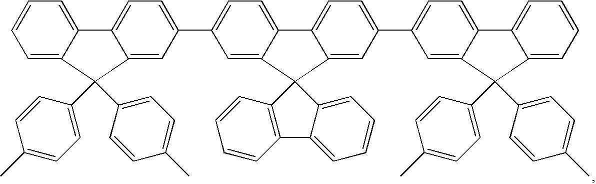
a sixth terfluorene compound having a chemical structure of 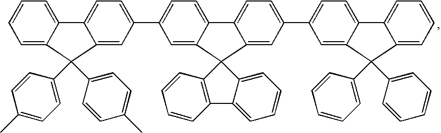
a seventh terfluorene compound having a chemical structure of 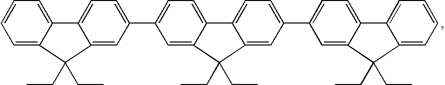
and a silole derivative (PyPySPyPy) compound having a chemical structural of [chemical structure]
